(19) United States
(12) Patent Application Publication
(10) Pub. No.: US 2003/0109757 A1
(43) Pub. Date: Jun. 12, 2003

(54) Method for separation and recovery of propargyl alcohol

(76) Inventors: Hideki Omori, Ichihara-shi (JP); Goro Sawada, Ichihara-shi (JP); Hideo Fukuda, Ichihara-shi (JP); Tomohiko Sato, Ichihara-shi (JP); Mitsuru Takeuchi, Ichihara-shi (JP)

Correspondence Address: Melvin L Stoltz, 51 Cherry Street, Milford, CT, 06460, US

(21) Appl. No.: 10/275,999

(22) PCT Filed: Mar. 18, 2002
(86) PCT No.: PCT/JP02/02541

(30) Foreign Application Priority Data
Mar. 21, 2001 (JP) 2001-080485

(51) Int. Cl. C07C 27/18; B01D 3/34

(52) U.S. Cl. 568/879; 203/38

(57) ABSTRACT

The present invention provides a method for separating and recovering propargyl alcohol from a product mixture containing a solvent, water and propargyl alcohol, which alleviates the problems of prior art and which can separate and recover propargyl alcohol in a simple operation at an advantageous thermal energy without requiring a large distillation unit or a complicated separation operation or step.

The method for separation and recovery of propargyl alcohol according to the present invention is characterized by subjecting, to distillation at a pressure of 100 to 150 mmHg, a propargyl alcohol-containing product mixture obtained by reacting paraformaldehyde with acetylene in the presence of a catalyst in a polar solvent.

### TECHNICAL FIELD

The present invention relates to a method for separating and recovering propargyl alcohol from a product mixture containing propargyl alcohol obtained by a reaction between paraformaldehyde and acetylene, particularly to a method for simply and efficiently separating propargyl alcohol from the solvent, etc. used in the reaction.

### BACKGROUND ART

For example of a synthesis of propargyl alcohol, a method which comprises subjecting an aldehyde or a ketone to a reaction with an acetylene type hydrocarbon in a particular solvent, in the presence of an alkali metal oxide or an alkali metal alcoholate as a catalyst, is known (U.S. Pat. No. 2,996,552). The product mixture after the reaction contains a large amount of the polar solvent and water which is contained in the raw materials or formed by the reaction; therefore, their separation from propargyl alcohol is necessary. However, it is not generally easy to separate propargyl alcohol of high polarity from the solvent of high polarity and water, by means of a distillation or the like and, particularly when the difference in boiling point between the solvent and propargyl alcohol is small, their separation is more difficult.

Hence, in order to separate propargyl alcohol, for example, a method which comprises adding fresh water of which separation inherently is difficult, to the product mixture and subjecting the resulting mixture to distillation to separate an azeotropic mixture of propargyl alcohol and water from the solvent, and a method which comprises adding a solvent showing azeotropy with water and subjecting the mixture to distillation to separate and recover propargyl alcohol, have hitherto been employed (U.S. Pat. No. 3,097,147). With such methods, however, it is apparent that a large distillation unit is required, the steps of distillation and separation are complicated, and there is a disadvantage in thermal energy.

Hence, the present invention aims at providing a method for separating and recovering propargyl alcohol from a product mixture containing a large amount of a solvent, water and propargyl alcohol as an intended product, which can separate and recover propargyl alcohol in a simple operation at an advantageous thermal energy without requiring a large distillation unit or a complicated separation operation or step and further without addition of other component which is disadvantageous in thermal energy.

The present inventors made a study in order to achieve the above aim. As a result, the present inventors paid attention to the distillation properties of propargyl alcohol and the reaction solvent used (e.g. dimethyl sulfoxide) and found out that by selecting particular distillation conditions, separation of propargyl alcohol from a product mixture can be conducted simply and efficiently. The present inventors made a further study and-completed the present invention.

### DISCLOSURE OF THE INVENTION

The gist of the present invention lies in a method for separation and recovery of propargyl alcohol, characterized by subjecting, to distillation at a pressure of 100 to 150 mmHg, a propargyl alcohol-containing product mixture obtained by reacting paraformaldehyde with acetylene in the presence of a catalyst in a polar solvent.

### BEST MODE FOR CARRYING OUT THE INVENTION

The present invention is described in detail below.

In the first stage of the present invention, first, paraformaldehyde is reacted with acetylene in the presence of a catalyst in a polar solvent to produce a propargyl alcohol-containing product mixture. In this case, an alkali metal hydroxide or the like, for example, sodium hydroxide or potassium hydroxide is used as the catalyst.

As to the amount of the alkali metal hydroxide used as the catalyst, an amount too small relative to paraformaldehyde as a raw material results in an increased amount of a by-product; conversely, too large an amount is neither advantageous nor economical. Therefore, the amount of the alkali metal hydroxide used is preferably 0.1 to 1.0 mole, particularly preferably 0.15 to 0.5 mole relative to 1 mole of formaldehyde as a raw material.

The polar solvent used in the above reaction of the present invention is preferably an aprotic polar solvent having a boiling point higher than that of propargyl alcohol. For example, dimethyl sulfoxide, dimethyl formamide or N-methylpyrrolidone can be used. Use of dimethyl sulfoxide is preferred from the standpoint of the yield of intended product.

The amount of the polar solvent used need not be very strict and can be selected desirably as long as the amount is at least a level capable of dispersing paraformaldehyde as a raw material and the catalyst and does not dilute the raw materials and the catalyst to such an extent that the reaction rate is reduced significantly.

One of the main raw materials required in production of propargyl alcohol by the above reaction is paraformaldehyde, and it is represented by the following general formula (1):

HOCH₂O(CH₂O)ₙCH₂OH (1)

wherein n is an integer of 1 to 100.

A paraformaldehyde preferred as a raw material in the above reaction is a paraformaldehyde which has, as n, 5 or 6 to less than 100 and is solid at room temperature (such a paraformaldehyde is a usual commercial product), because it contains water in a small amount.

The remaining main raw material required in production of propargyl alcohol by the above reaction is acetylene. The acetylene may comprise a commercial product filled in a gas cylinder and also a product per se obtained by subjecting an acetylene contained in an ethylene fraction obtained from a naphtha cracker, to extraction with a polar solvent such as dimethyl formamide or the like and subsequent recovery.

The reaction for synthesis of propargyl alcohol, in the present invention can be conducted continuously or batchwise. When it is conducted continuously as shown in Example shown later, for example, first, a polar solvent and acetylene are placed in this order in a reactor and they are kept at a predetermined temperature with stirring. Then, a reaction is started with a paraformaldehyde slurry (a dispersion in a polar solvent) and a catalyst slurry (a dispersion in a polar solvent) both being introduced continuously. Simultaneously with the introduction of these components or after the reaction has proceeded for a certain length of time, the product mixture is taken out continuously while the liquid phase level in the reactor is kept constant.

In the above reaction, the reaction temperature is preferably 0 to 100° C., more preferably 10 to 60° C., and the reaction pressure in terms of acetylene partial pressure is preferably 0 to 1 MPa (a gauge pressure), more preferably 0 to 0.20 MPa (a gauge pressure).

In the above reaction, a higher acetylene partial pressure gives a higher reaction rate but tends to cause decomposition and explosion of acetylene; therefore, a low acetylene partial pressure is desired in order to prevent the decomposition and explosion. Therefore, the reaction may be conducted by introducing an inert gas such as nitrogen, argon, propane or the like to dilute acetylene.

The product mixture after the reaction is subjected to the second stage of the present invention, i.e. a step for removal of the alkali metal hydroxide (a catalyst) contained therein and successively to the third stage, i.e. a step for separation of reaction solvent, etc. The second stage, i.e. the step for removal of the alkali metal hydroxide (a catalyst) is conducted by first separating solid components by filtration, centrifugation or the like and, as to the remaining alkali metal hydroxide, adding water to the product mixture and conducting extraction and separation, or adding an acidic compound such as carbon dioxide or the like for neutralization and separating the resulting salt.

The solution recovered in the above step contains propargyl alcohol (an intended product), the polar solvent, water and generally a small amount of paraformaldehyde and a small amount of 1,4-butynediol which is a by-product.

Then, the recovered solution is subjected to a distillation step which is the third step, to separate propropargyl alcohol from a large amount of the polar solvent, etc. in the product mixture. The distillation conditions are determined in view of the distillation properties of propargyl alcohol and the reaction solvent.

For example, when dimethyl sulfoxide is used as the polar solvent, the temperature in distillation is basically not higher than 130° C. (the thermal decomposition temperature of dimethyl sulfoxide) because dimethyl sulfoxide causes thermal decomposition easily; the pressure in distillation is not higher than a pressure corresponding to the distillation temperature of 130° C. (the thermal decomposition temperature of dimethyl sulfoxide).

With dimethyl sulfoxide, however, as the pressure in distillation is lower, the relative volatility (α) between propargyl alcohol (a low-boiling component) and dimethyl sulfoxide (a high-boiling component), represented by the following formula (I) tends to be smaller; therefore, it is preferred to use a pressure as high as possible in a temperature range in which dimethyl sulfoxide causes no decomposition. The reason is that while the relative volatility (α) represented by the formula (I) is large when the molar fraction y of the low-boiling component in vapor is large or when the molar fraction x of the low-boiling component in solution is small, a higher pressure in distillation can make larger the relative volatility (α) represented by the general formula (I), as shown in the following Table 1, and can be advantageous to a separate efficiency of the low-boiling component in distillation.

α=[y/(1−y)]×[(1−x)/x] (I)

(In the above formula, x and y are, respectively, molar fractions of low-boiling component contained in solution and vapor in distillation system which are in equilibrium at a particular temperature.)

Meanwhile, when the low-boiling component coexisting with dimethyl sulfoxide is not propargyl alcohol but other compound such as benzene or the like, the relative volatility of the low-boiling component is generally smaller as the distillation pressure is higher, as shown in the following Table 2; therefore, no advantage in separation such as mentioned above is obtained. Thus, the above-mentioned behavior of a combination of dimethyl sulfoxide solvent and propargyl alcohol is specific.

TABLE 1
Propargyl alcohol-dimethyl sulfoxide system (106° C.)

| Pressure (mmHg) | Relative volatility (α) |
|---|---|
| 110 | 6.0 |
| 95 | 5.3 |
| 40 | 1.8 |

TABLE 2
Benzene-dimehtyl sulfoxide system (40° C.)

| Pressure (mmHg) | Relative volatility (α) |
|---|---|
| 103 | 186 |
| 80 | 236 |
| 49 | 285 |

Therefore, when dimethyl sulfoxide is used as a reaction solvent, the distillation pressure is preferred to be not higher than 150 mmHg which is the distillation pressure at 130° C. (the decomposition temperature of dimethyl sulfoxide) but be not so low, for example, 100 to 150 mmHg.

Examples of the type of the distillation column used may include a flash vaporization column, a plate column and a packed tower. Use of a rectifying column is preferred in order to obtain a separation efficiency as high as possible.

### EXAMPLES

The present invention is described in more detail by way of Examples and Comparative Examples. However, the present invention is not restricted thereto.

Incidentally, the analytical methods used in the following Example and Comparative Example are as follows.

(1) The reaction products were analyzed by gas chromatography.

(2) The amount of paraformaldehyde determined by iodometry compressing a reaction with iodine under an alkali condition and a titration with a solution of sodium thiosulfate in the presence of starch as an indicator.

(3) The amount of water was determined by the Karl Fischer's method.

### Example 1

(1) Reaction Between Paraformaldehyde and Acetylene (First Stage)

4 liters of dimethyl sulfoxide and acetylene were introduced into an autoclave having an internal volume of 10 liters. The autoclave inside was kept at a pressure (a gauge pressure) of 0.02 MPa. Then, a slurry of 16.2% by weight of a paraformaldehyde of the general formula (1) wherein n was 8 to 9, dispersed in dimethyl sulfoxide and a slurry of 7.2% by weight of a potassium hydroxide dispersed in dimethyl sulfoxide were continuously fed, at rates of 414.5 g/hr and 374.5 g/hr, respectively. Then, a reaction was allowed to take place at a reaction temperature of 25° C. at an acetylene partial pressure (a gauge pressure) of 0.02 MPa. Part of the resulting product mixture was continuously taken out from the reaction system so that the liquid phase level inside the reactor was kept constant, and the product mixture taken out was analyzed. 18 hours later, it was confirmed that a steady state was reached, and the product mixture showed a composition of 72 g/hr of propargyl alcohol, 11 g/hr of 1,4-butinediol, 695 g/hr of dimethyl sulfoxide, 21 g/hr of paraformaldehyde, 4 g/hr of water and 27 g/hr of potassium hydroxide.

(2) Removal of Potassium Hydroxide (Second Stage)

Then, the product mixture taken out was neutralized with carbon dioxide gas, and the resulting solid was removed by filtration.

The filtrate obtained, when analyzed, contained 9.1% by weight of propargyl alcohol, 1.4% by weight of 1,4-butinediol, 0.6% by weight of paraformaldehyde, 87.9% by weight of dimethyl sulfoxide and 1.0% by weight of water.

(3) Distillation of Filtrate (Third Step)

The filtrate obtained above was fed into a distillation column of 15 plates, and continuous distillation was conducted at a pressure of 110 mmHg. When the bottom temperature of the distillation column reached 127° C. and the top temperature reached 60° C., a distillate containing 85.9% by weight of propargyl alcohol, 0.7% by weight of dimethyl sulfoxide, 2.8% by weight of paraformaldehyde and 10.5% by weight of water was obtained from the top of the distillation column. Meanwhile, from the bottom of the distillation column was obtained a bottom product containing 97.1% by weight of dimethyl sulfoxide, 1.0% by weight of propargyl alcohol, 0.4% by weight of paraformaldehyde and 1.5% by weight of 1,4butinediol. From this result, it is appreciated that the dimethyl sulfoxide solvent could be removed almost completely under the conditions of the present test.

### Comparative Example 1

Paraformaldehyde and acetylene were reacted in the same manner as in Example 1. Successively, catalyst removal and filtration were conducted to obtain a filtrate consisting of 9.1% by weight of propargyl alcohol, 1.4% by weight of 1,4-butinediol, 0.6% by weight of paraformaldehyde, 87.9% by weight of dimethyl sulfoxide and 1.0% by weight of water.

The filtrate was fed into the same distillation as in Example 1 and distillation was conducted at a pressure of 13.5 mmHg. No distillate was obtained at the boiling point (about 16° C.) of propargyl alcohol, and a distillate appeared at a column top temperature of 73° C. (about the boiling point of dimethyl sulfoxide) (at this time, the column bottom temperature was 92° C.). The ratio of propargyl alcohol and dimethyl sulfoxide in the distillate was the same as the ratio in fed solution, and they were not separated at all.

Industrial Applicability

As seen from the results of the above Example and Comparative Example, in the method of the present invention for separating and recovering propargyl alcohol from a product mixture containing a large amount of a solvent, water and propargyl alcohol as an intended product, propargyl alcohol can be separated and recovered in a simple operation at an advantageous thermal energy without requiring a large distillation unit or a complicated separation operation or step and further without addition of other component, which is disadvantageous in thermal energy.

1. A method for separation and recovery of propargyl alcohol, characterized by subjecting, to distillation at a pressure of 100 to 150 mmHg, a propargyl alcohol-containing product mixture obtained by reacting paraformaldehyde with acetylene in the presence of a catalyst in a polar solvent.

2. A method for separation and recovery of propargyl alcohol according to claim 1, wherein the polar solvent is dimethyl sulfoxide.

3. A method for separation and recovery of propargyl alcohol according to claim 1 or 2, wherein the reaction of paraformaldehyde with acetylene is conducted at 0 to 100° C. at an acetylene partial pressure of 0 to 1 MPa (a gauge pressure).

4. A method for separation and recovery of propargyl alcohol according to claim 1, wherein catalyst removal is conducted prior to the distillation.
